(19) United States
(12) Patent Application Publication
(10) Pub. No.: US 2011/0130814 A1
(43) Pub. Date: Jun. 2, 2011

(54) Air permeable member for portable body warmer

(75) Inventors: Masaki Nagano, Matsuyama-shi (JP); Yasuhiro Takeda, Matsuyama-shi (JP)

(73) Assignee: NITTO LIFETEC CORPORATION, Matsuyama-shi (JP)

(21) Appl. No.: 12/801,662

(22) Filed: Jun. 18, 2010

(30) Foreign Application Priority Data
Nov. 30, 2009 (JP) 2009-272040

(51) Int. Cl. A61F 7/08

(52) U.S. Cl. 607/114

(57) ABSTRACT

An air-permeable member for portable body warmers includes a nonwoven fabric and a porous film in a layered structure. The nonwoven fabric is a spunbonded nonwoven fabric having an embossment area ratio of 5% to 20% and a METSUKE of 10 to 80 g/m², the porous film has a thickness of 30 to 200 μm, and the air-permeable member has a compressive strength in machine direction of from 2 to 5.5 N as a maximum compressive load measured through a ring crush method by sampling a 60-mm square test piece; rounding the test piece with a circumference agreeing the transverse direction to give a cylindrical sample; compressing the cylindrical sample in the machine direction using a tensile tester at a temperature of 23° C. and relative humidity of 50% at a compressing speed of 300 mm/min.; and measuring compressive loads at distortions of 0 to 10 mm.

### BACKGROUND OF THE INVENTION

1. Field of the Invention

The present invention relates to members having air permeability (air-permeable members) to be adopted to portable body warmers. More specifically, it relates to air-permeable members which can give such portable body warmers that are resistant to deformation and are usable with comfortable feeling without unpleasantness.

2. Description of the Related Art

Certain portable body warmers such as disposable (single-use) portable body warmers each include a heater (exothermic material) and a bag housing the heater. Exemplary currently known members for constituting the bag (bag-constituting members) include air-permeable materials (air-permeable members) each including a porous film and an air-permeable base material such as a nonwoven fabric bonded with each other (see, for example, Japanese Unexamined Patent Application Publication (JP-A) No. H08 (1996)-131472; Japanese Unexamined Patent Application Publication (JP-A) No. H10 (1998)-314208; Japanese Unexamined Patent Application Publication (JP-A) No. 2000-42021; and Japanese Unexamined Patent Application Publication (JP-A) No. 2000-126217).

Such portable body warmers have to give comfortable feeling without unpleasantness when used while affixing typically to clothing or skin.

### SUMMARY OF THE INVENTION

Some portable body warmers give improved feeling upon use by using flexible air-permeable members typically including spunlace nonwoven fabrics made typically from nylons or poly(ethylene terephthalate)s (PETs). These portable body warmers, however, tend to deform and suffer from low productivity such that the portable body warmers deform during transfer in production processes of portable body warmers to cause transfer failure.

Accordingly, an object of the present invention is to provide an air-permeable member for portable body warmers, which member gives a portable body warmer being usable with comfortable feeling without unpleasantness and being resistant to deformation.

After intensive investigations, the present inventors have found that a specific air-permeable member gives a portable body warmer which is suppressed from deforming typically in production processes and which is protected from unpleasantness upon use thereof. This air-permeable member is an air-permeable member for portable body warmers, which has a layered structure with a porous film having a specific thickness and a spunbond nonwoven fabric having a specific embossment area ratio and a specific mass per unit area and which has a controlled compressive strength in a machine direction as determined according to a ring crush method within a specific range. The present invention has been made based on these findings.

Specifically, the present invention provides, in an embodiment, an air-permeable member for portable body warmers, which member has a layered structure including at least a nonwoven fabric and a porous film, in which the nonwoven fabric is a spunbonded nonwoven fabric having an embossment area ratio of from 5% to 20% and a mass per unit area of from 10 to 80 g/m², the porous film has a thickness of from 30 to 200 μm, and the air-permeable member has a compressive strength in a machine direction of from 2 to 5.5 newtons (N) as measured according to a ring crush method. The “compressive strength in a machine direction as measured according to the ring crush method” is a maximum compressive load determined by sampling a 60-mm square test piece from the air-permeable member for portable body warmers, the test piece having sides corresponding to the machine direction and transverse direction of the air-permeable member, respectively; rounding the test piece with a circumference agreeing the transverse direction to give a cylindrical measurement sample; compressing the cylindrical measurement sample in the machine direction at an ambient temperature of 23° C. and relative humidity of 50% at a compressing speed of 300 mm per minute using a tensile tester according to a compression mode; and measuring compressive loads at distortions (compression distances) of from 0 to 10 mm to determine the maximum compressive strength.

The present invention further provides, in another embodiment, an air-permeable member for portable body warmers, which member has a layered structure including at least a nonwoven fabric and a porous film, in which the nonwoven fabric is a composite nonwoven fabric including a spunbonded nonwoven fabric and a spunlace nonwoven fabric, the spunbonded nonwoven fabric having an embossment area ratio of from 5% to 20% and a mass per unit area of from 10 to 80 g/m², the porous film has a thickness of from 30 to 200 μm, and the air-permeable member has a compressive strength in a machine direction of from 2 to 5.5 N as measured according to the ring crush method as above.

The nonwoven fabric herein preferably includes a polyester nonwoven fabric.

The air-permeable member for portable body warmers according to the present invention uses a spunbond nonwoven fabric having an embossment area ratio and a mass per unit area controlled within specific ranges; and a porous film having a specific thickness. In addition, the air-permeable member has a compressive strength in the machine direction as measured according to the ring crush method controlled within a specific range. The air-permeable member is advantageous to form a portable body warmer which is resistant to deformation and is usable comfortably without unpleasantness, whereby the resulting portable body warmer has improved productivity and quality.

### BRIEF DESCRIPTION OF THE DRAWINGS

Other objects, features and advantages of the present invention will be understood more fully from the following detailed description made with reference to the accompanying drawings. In the drawings:

FIG. 1 is a schematic diagram (cross-sectional view) illustrating an air-permeable member for portable body warmers, according to an embodiment of the present invention;

FIG. 2 is a schematic diagram (cross-sectional view) illustrating an air-permeable member for portable body warmers, according to another embodiment of the present invention;

FIG. 3 is a schematic diagram (cross-sectional view) illustrating an exemplary portable body warmer using the air-permeable member for portable body warmers according to an embodiment of the present invention;

FIG. 4 is a schematic diagram (perspective view) showing a cylindrical measurement sample for use in the measurement of the compressive strength (ring crush method, MD); and

FIG. 5 is an explanatory drawing schematically illustrating how to set the measurement sample in a tensile tester in the measurement of the compressive strength (ring crush method, MD) and is a schematic front view showing a region around grips (chucks) of the tensile tester.

### DESCRIPTION OF THE PREFERRED EMBODIMENTS

Air-permeable members for portable body warmers according to embodiments of the present invention (hereinafter also simply referred to as “air-permeable members according to the present invention” or “air-permeable members”) are air-permeable members each including a composite material (composite member) having a layered structure including at least a nonwoven fabric (nonwoven fabric layer) and a porous film (porous film layer). The nonwoven fabric and the porous film are preferably bonded with each other through an adhesive layer and are more preferably bonded with each other through a porous adhesive layer formed from a fiberized adhesive.

Nonwoven Fabric (Nonwoven Fabric Layer)

Though not especially limited, the nonwoven fabrics (nonwoven fabric layers) in the air-permeable members are preferably polyester-based nonwoven fabrics made from polyester fibers (polyester nonwoven fabrics). Such polyester nonwoven fabrics are preferred, because, when used as the nonwoven fabric, they show high “rigidity”, and this enables easier control of the compressive strength in a machine direction (MD) as measured according to the ring crush method (hereinafter also referred to as “compressive strength (ring crush method, MD)”) of the air-permeable members within the range (2 to 5.5 N) specified herein; and this in turn helps portable body warmers formed from the air-permeable members to be more resistant to deformation. Of the polyester nonwoven fabrics, preferred examples from the viewpoint of higher rigidity are PET nonwoven fabrics including polyethylene terephthalate) (PET) fibers; PBT nonwoven fabrics including poly(butylene terephthalate) (PBT) fibers; and nonwoven fabrics including both PET fibers and PBT fibers. Each of nonwoven fabrics may include fibers of one type or fibers of two or more types. The fiber diameters, fiber lengths, and other conditions in the nonwoven fabrics are not especially limited.

The nonwoven fabrics (nonwoven fabric layers) in the air-permeable members each include, as an essential nonwoven fabric, a spunbonded nonwoven fabric having an embossment area ratio of from 5% to 20% and a mass per unit area of from 10 to 80 g/m². Herein a nonwoven fabric prepared by spunbonding (spunbond technique) is also referred to as “spunbond nonwoven fabric”; a nonwoven fabric prepared by spunlacing (spunlace technique) is also referred to as a “spunlace nonwoven fabric”; and the “spunbonded nonwoven fabric having an embossment area ratio of from 5% to 20% and a mass per unit area of from 10 to 80 g/m²” is also referred to as a “nonwoven fabric (a)” or “spunbond nonwoven fabric (a)”.

The nonwoven fabrics (nonwoven fabric layers) in the air-permeable members may each be a nonwoven fabric (a) [i.e., a nonwoven fabric including the nonwoven fabric (a) alone] or a composite nonwoven fabric (laminate of nonwoven fabrics) including the nonwoven fabric (a) and one or more other nonwoven fabrics.

The nonwoven fabric (a) is a spunbond nonwoven fabric prepared through spunbonding. The nonwoven fabric, as being a spunbond nonwoven fabric, has higher “rigidity”. This allows the air-permeable members to have higher “rigidity” and to have a controlled compressive strength (ring crush method, MD) within the range specified herein. A spunlace nonwoven fabric, if adopted alone, does not help air-permeable members to have sufficiently high compressive strengths (ring crush method, MD), and portable body warmers made from them are liable to deform and have insufficient productivity.

The nonwoven fabric (a) is preferably an embossed spunbond nonwoven fabric and is especially preferably an embossed spunbond nonwoven fabric embossed with a hot embossing roll. The embossment pattern is not especially limited in its shape and may for example be rectangular or circular. Though not critical, the area per single embossment pattern is preferably from 0.1 to 10 mm², and more preferably from 0.3 to 5 mm². The control of the area per single embossment pattern within the above range makes it easier to control the compressive strength (ring crush method, MD) of the air-permeable members within the range specified herein. If the area is less than 0.1 mm², the area may be too small to give a sufficient strength as a result of heat sealing; and if it exceeds 10 mm², the embossments may be arranged at excessively large intervals; and in the both cases, the air-permeable members may not have sufficient compressive strengths (ring crush method, MD).

The embossment area ratio of the nonwoven fabric (a) is 5% to 20%, preferably 7% to 20%, and more preferably from 7% to 17%. The control of the embossment area ratio to 5% or more helps the nonwoven fabric (a) and the air-permeable members according to the present invention to have higher “rigidity”, and this controls the compressive strength (ring crush method, MD) of the air-permeable members to 2 N or more. The control of the embossment area ratio to 20% or less prevents the nonwoven fabric (a) and the air-permeable members according to the present invention from being excessively rigid, and this controls the compressive strength (ring crush method, MD) of the air-permeable members to 5.5 N or less.

The embossment area ratio (also referred to as “embossed area ratio”) refers to the ratio of the area of embossed regions to the total area of the nonwoven fabric. The embossment area ratio can be determined by measuring the ratio of the area of the embossed region in the measurement area with a microscope.

The mass per unit area (METSUKE) of the nonwoven fabric (a) is from 10 to 80 g/m², preferably from 20 to 50 g/m², and more preferably from 20 to 40 g/m², from the viewpoint of controlling the compressive strength (ring crush method, MD) of the air-permeable members within the range specified herein. The control of the mass per unit area to 10 g/m² or more helps the nonwoven fabric (a) and the air-permeable members to have higher “rigidity”, and this helps to control the compressive strengths (ring crush method, MD) of the air-permeable members to 2 N or more. The control of the mass per unit area to 80 g/m² or less suppresses the nonwoven fabric (a) and the air-permeable members from being excessively rigid, and this helps to control the compressive strengths (ring crush method, MD) of the air-permeable members to 5.5 N or less. In addition, if the mass per unit area is excessively low of less than 10 g/m², the nonwoven fabric has an inferior appearance due to an excessively low fiber density; and if it is excessively high of more than 80 g/m², the nonwoven fabric becomes a high-cost product.

Though not critical, the compressive strength (ring crush method, MD) of the nonwoven fabric (a) alone is preferably from 0.1 to 4.0 N, and more preferably from 0.1 to 3.0 N, from the viewpoint of controlling the compressive strength (ring crush method, MD) of the air-permeable members within the range specified herein.

The nonwoven fabric (a) can be prepared according to a known or common spunbonding technique (spunbond technique). The nonwoven fabric (a) can also be any of commercially available spunbond nonwoven fabrics, as long as having a mass per unit area and an embossment area ratio within the specific ranges, of which polyester spunbond nonwoven fabrics are preferred.

When the nonwoven fabric (nonwoven fabric layer) in the air-permeable members is a composite nonwoven fabric including a nonwoven fabric (a) with one or more other nonwoven fabrics, the “other nonwoven fabrics” are not especially limited but are preferably spunlace nonwoven fabrics. By using a composite nonwoven fabric including a nonwoven fabric (a) and a spunlace nonwoven fabric and using the spunlace nonwoven fabric so as to face outward (as front side), the air-permeable members have more satisfactory feel and texture. Though not critical, the composite nonwoven fabric preferably has a two-layer structure of (nonwoven fabric (a))/(other nonwoven fabric). The composite nonwoven fabric having a two-layer structure of (nonwoven fabric (a))/(other nonwoven fabric) is preferably used in the air-permeable members so that the nonwoven fabric (a) faces the porous film, and the other nonwoven fabric is present opposite to the porous film (i.e., present as the front side of air-permeable member).

Though not critical, the mass per unit area of the other nonwoven fabric is preferably from 10 to 50 g/m², and more preferably from 10 to 30 g/m², from the viewpoint of cost.

The other nonwoven fabric can be prepared according to any of known or common production processes of nonwoven fabrics, of which spunlacing is preferred. The other nonwoven fabric can also be any of commercially available nonwoven fabrics, of which polyester spunlace nonwoven fabrics are preferred. Though not especially limited, the way to compound the nonwoven fabric (a) with the other nonwoven fabric may be a process of compounding a spunbond nonwoven fabric (nonwoven fabric (a)) with another nonwoven fabric (for example, spunlace nonwoven fabric) through hydroentangling.

Though not critical, the compressive strength (ring crush method, MD) of the composite nonwoven fabric (nonwoven fabric layer), when used as the nonwoven fabric in the air-permeable members, is preferably from 0.1 to 4.0 N, and more preferably from 0.1 to 3.0 N. This range is preferred from the viewpoint of controlling the compressive strength (ring crush method, MD) of the air-permeable members within the range specified herein.

Of such nonwoven fabrics, the nonwoven fabrics (nonwoven fabric layer) in the air-permeable members are each preferably a polyester nonwoven fabric (a) [i.e., a nonwoven fabric including the nonwoven fabric (a) alone] or a composite nonwoven fabric of a polyester nonwoven fabric (a) with a polyester spunlace nonwoven fabric.

Porous Film (Porous Film Layer)

The porous films (porous film layers) in the air-permeable members are film-shaped porous base materials made from, for example, any of olefinic resins, polyester resins, and styrenic resins. Of these, porous films made from olefinic resins are preferably used from the viewpoints typically of price, flexibility, and heat sealability.

The olefinic resins for use herein are not especially limited, as long as being resins containing at least one olefin component as a monomer component. Exemplary olefin components include α-olefins such as ethylene, propylene, butene-1, pentene-1, hexene-1,4-methyl-pentene-1, heptene-1, and octene-1.

Exemplary olefinic resins include ethylenic resins such as low-density polyethylenes, linear low-density polyethylenes, medium-density polyethylenes, high-density polyethylenes, ethylene-vinyl acetate copolymers, and ethylene-α-olefin copolymers (for example, ethylene-propylene copolymers); propylene resins such as polypropylenes and propylene-α-olefin copolymers; polybutene resins such as polybutene-1; and poly-4-methylpentene-1. Exemplary olefinic resins usable herein further include ethylene-unsaturated carboxylic acid copolymers such as ethylene-acrylic acid copolymers and ethylene-methacrylic acid copolymers; ionomers; ethylene-(meth)acrylic ester copolymers such as ethylene-methyl acrylate copolymers, ethylene-ethyl acrylate copolymers, and ethylene-methyl methacrylate copolymers; and ethylene-vinyl alcohol copolymers. Of the olefinic resins, ethylenic resins are preferred, of which low-density polyethylenes, linear low-density polyethylenes, and ethylene-α-olefin copolymers are more preferred.

The densities of the low-density polyethylenes are preferably from 0.90 to 0.93 g/cm³, and more preferably from 0.91 to 0.92 g/cm³. Though not critical, the weight-average molecular weights of the low-density polyethylenes are preferably from 3×10⁴ to 20×10⁴, and more preferably from 5×10⁴ to 6×10⁴. Also though not critical, the melt flow rates (MFRs) of the low-density polyethylenes at 190° C. are preferably from 1.0 to 5.0 (grams per 10 minutes (g/10 min.)), and more preferably from 2.0 to 4.0 (g/10 min.). As used herein the term “density” refers to a density determined in accordance with Japanese Industrial Standards (JIS) K 6922-2 and JIS K 7112. The MFRs herein can be measured in accordance with International Organization for Standardization (ISO) Standards 1133 (corresponding to JIS K 7210). The weight-average molecular weights can be measured through a gel permeation chromatography (GPC) technique. Of such gel permeation chromatography techniques, high-temperature gel permeation chromatography using a high-temperature gel permeation chromatograph is preferably adopted. Specifically, the high-temperature gel permeation chromatography described in Japanese Unexamined Patent Application Publication (JP-A) No. 2009-184705 can for example be adopted.

The linear low-density polyethylenes are linear polyethylenes prepared through polymerization of ethylene with an α-olefin monomer having 4 to 8 carbon atoms and having short-chain branches. The branches preferably have 1 to 6 carbon atoms in length. Preferred α-olefin monomers for use in the linear low-density polyethylenes include 1-butene, 1-octene, 1-hexene, and 4-methylpentene-1. In the linear low-density polyethylenes, the content (percentage content) of ethylene monomer repeating units (repeating units derived from ethylene monomer) is preferably 90 percent by mole or more of the total repeating units of total constituent monomers (repeating units derived from all the constituent monomers). Of the linear low-density polyethylenes, especially preferred are so-called metallocene-catalyzed linear low-density polyethylenes (metallocene-catalyzed LLDPEs) prepared by using a metallocene catalyst, because they contribute to the improvements in heat sealability at lower temperatures.

The densities of the linear low-density polyethylenes are preferably from 0.90 to 0.93 g/cm³, and more preferably from 0.91 to 0.92 g/cm³. Though not critical, the weight-average molecular weights of the linear low-density polyethylenes are preferably from 3×10⁴ to 20×10⁴, more preferably from 5×10⁴ to 10×10⁴, and furthermore preferably from 5×10⁴ to 6×10⁴. Also though not critical, the MFRs of the linear low-density polyethylenes at 190° C. are preferably from 1.0 to 5.0 (g/10 min.), and more preferably from 2.0 to 4.0 (g/10 min.).

The ethylene-α-olefin copolymers are copolymers of ethylene and an α-olefin monomer. The α-olefin is not especially limited, as long as being an α-olefin other than ethylene. Exemplary α-olefins include α-olefins having 3 to 8 carbon atoms, such as propylene, butene-1, pentene-1, hexene-1,4-methyl-pentene-1, heptene-1, and octene-1; of which ethylene-α-olefin elastomers (copolymers) using butene-1 are preferred. In the ethylene-α-olefin copolymers, the content of ethylene monomer repeating units is preferably from 60 to 95 percent by mole, and more preferably from 80 to 90 percent by mole, of the moles of repeating units of the total constituent monomers. The ethylene-α-olefin copolymers play a role of imparting further satisfactory heat sealability to the porous film.

The densities of the ethylene-α-olefin copolymers are preferably less than 0.90 g/cm³, more preferably from 0.86 to 0.89 g/cm³, and furthermore preferably from 0.87 to 0.89 g/cm³. Though not critical, the weight-average molecular weights of the ethylene-α-olefin copolymers are preferably from 5×10⁴ to 20×10⁴, and more preferably from 8×10⁴ to 15×10⁴. Also though not critical, the MFRs of the ethylene-α-olefin copolymers at 190° C. are preferably from 1.0 to 5.0 (g/10 min.), and more preferably from 2.0 to 4.0 (g/10 min.).

The porous film for use herein is preferably, but is not limited to, a porous film containing one or more inorganic fillers. The inorganic fillers form voids (holes) around them as a result of drawing and thereby play a role of rendering the film to be porous. Exemplary inorganic fillers include talc, silica, stone powder, zeolite, alumina, aluminum powder, and iron powder; as well as metal carbonates such as calcium carbonate, magnesium carbonate, calcium/magnesium carbonate, and barium carbonate; metal sulfates such as magnesium sulfate and barium sulfate; metal oxides such as zinc oxide, titanium oxide, and magnesium oxide; metal hydroxides such as aluminum hydroxide, magnesium hydroxide, zirconium hydroxide, calcium hydroxide, and barium hydroxide; and metal hydrates (hydrated metallic compounds) such as a hydrate of magnesium oxide and nickel oxide, and a hydrate of magnesium oxide and zinc oxide. Among them, calcium carbonate and barium sulfate are preferred. Though not critical, the inorganic fillers may be, for example, tabular or granular in their shapes, but are preferably granular (particle-form) so as to form voids (holes) more satisfactorily as a result of drawing. Specifically, inorganic particles (inorganic microparticles) including calcium carbonate are preferred as the inorganic fillers.

Though not critical, the particle diameter (average particle diameter) of the inorganic fillers (inorganic particles) is, for example, preferably from 0.1 to 10.0 μm, and more preferably from 0.5 to 5.0 μm. This range is preferred because inorganic fillers, when having a particle diameter of 0.1 μm or more, may more effectively contribute to the formation of voids; and, when having a particle diameter of 10.0 μm or less, they may help to suppress the breakage during film formation and to avoid inferior appearance.

Though not critical, the content of inorganic fillers (inorganic particles) is preferably from 50 to 150 parts by weight, and more preferably from 80 to 120 parts by weight, typically per 100 parts by weight of the total polymer components constituting the porous film. Inorganic fillers, when present in a content of 50 parts by weight or more, may more effectively contribute to the formation of voids; and, when present in a content of 150 parts by weight or less, they may help to suppress the breakage during film formation and to avoid inferior appearance.

The porous film for use herein may further contain additives within ranges not adversely affecting the advantages of the present invention. Exemplary additives herein include colorants, age inhibitors, antioxidants, ultraviolet-absorbers, flame retardants, and stabilizers.

The porous film for use herein can be prepared by a film formation process using melting state, such as T-die process or tubular film process. Of such processes, T-die process is preferred. Typically, the porous film may be prepared by mixing and dispersing the olefinic resin, inorganic filler, and, according to necessity, additives using a twin-screw kneader/extruder to give pellets, and melting the pellets and extruding the molten pellets in a single-screw extruder to give an unoriented film, and drawing the unoriented film monoaxially or biaxially to give a porous film. A multilayer film, when used as the porous film, is preferably prepared through coextrusion. Where necessary, the porous film may be subjected to one or more treatments such as backing and antistatic treatment.

In the preparation of the porous film, the extrusion is performed at a temperature of preferably from 170° C. to 270° C., more preferably from 180° C. to 260° C., and furthermore preferably from 230° C. to 250° C. The haul-off of the unoriented film is performed at a speed of preferably from 5 to 25 meters per minute and at a haul-off roll temperature (cooling temperature) of preferably from 5° C. to 40° C., and more preferably from 20° C. to 30° C.

The monoaxial or biaxial drawing (sequential biaxial drawing or simultaneous biaxial drawing) of the unoriented film can be performed according to a known or common drawing procedure such as drawing using a roll or drawing using a tenter. The drawing temperature is preferably from 50° C. to 100° C., and more preferably from 60° C. to 90° C. The draw ratio (in one direction) is preferably from 2 to 5 times, and more preferably from 3 to 4 times, from the viewpoint of forming a satisfactorily porous film stably. The draw ratio by area in the biaxial drawing is preferably from 2 to 10 times, and more preferably from 3 to 7 times.

The thickness of the porous film is from 30 to 200 μm, preferably 50 to 150 μm, and more preferably from 50 to 120 μm. The porous film, as having a thickness controlled to be 30 μm or more, helps the air-permeable members to have a controlled compressive strength (ring crush method, MD) of 2 N or more. The porous film, as having a thickness controlled to be 200 μm or less, helps the air-permeable members to have a controlled compressive strength (ring crush method, MD) of 5.5 N or less.

Though not critical, the compressive strength (ring crush method, MD) of the porous film itself is preferably from 0.1 to 4.0 N, and more preferably from 0.2 to 3.0 N, from the viewpoint of controlling the compressive strengths (ring crush method, MD) of the air-permeable members within the range specified herein.

Adhesive (Adhesive Layer)

Though not limited, the nonwoven fabric (nonwoven fabric layer) and the porous film (porous film layer) in the air-permeable members are preferably laminated with each other by bonding through an adhesive layer. Specifically, the nonwoven fabric and the porous film are preferably bonded with each other through an adhesive layer. The adhesive layer is not especially limited, as long as not adversely affecting the air permeability of the air-permeable members and as long as controlling the compressive strength (ring crush method, MD) of the air-permeable members within the range specified herein. The adhesive layer can be a known adhesive layer for use typically in bonding of nonwoven fabrics with porous films. As used herein the “adhesive” for constituting the adhesive layer also includes a “pressure-sensitive adhesive (tacky adhesive)”.

The adhesive for constituting the adhesive layer can be, but is not limited to, any of known adhesives including rubber adhesives (such as natural rubbers and styrenic elastomers), urethane adhesives (acrylic urethane adhesives), polyolefinic adhesives (such as ethylene-vinyl acetate copolymers (EVAs) and ethylene-methyl acrylate copolymers (EMAs)), acrylic adhesives, silicone adhesives, polyester adhesives, polyamide adhesives, epoxy adhesives, vinyl alkyl ether adhesives, and fluorine-containing adhesives. Each of different adhesives can be used alone or in combination. Among them, polyamide adhesives and polyester adhesives are especially preferred.

Such adhesives for use herein can be adhesives of every form, of which hot-melt (thermofusible) adhesives are preferred, because they can be applied by heating and melting without the use of solvents, can be directly applied even to nonwoven fabrics to form an adhesive layer, and can give a further higher adhesive strength in the heat-sealed section as a result of heat sealing. Specifically, of the adhesives, polyamide or polyester hot-melt adhesives are preferred, of which thermoplastic polyamide hot-melt adhesives and thermoplastic polyester hot-melt adhesives are more preferred.

The specific way to laminate the nonwoven fabric with the porous film may vary depending typically on the type of the adhesive and is not particularly limited. Typically, when a hot-melt adhesive is used, the lamination is preferably carried out by applying the adhesive to a nonwoven fabric, and bonding a porous film therewith. The application can be carried out using any known or common procedure used for the application of hot-melt adhesives. Typically, the application is preferably carried out by spray coating, stripe coating, or dot coating, for maintaining the air permeability. Though not critical, the mass of coating (solids content) of adhesive is preferably from 0.5 to 20 g/m², and more preferably from 1 to 10 g/m², from the viewpoints of adhesion of the heat-sealed section and economical efficiency upon the formation of the bag.

Of such adhesive layers, a porous adhesive layer is especially preferred as the adhesive layer in the air-permeable members. Specifically, in a preferred embodiment, the air-permeable member is a laminate (composite material) including a nonwoven fabric (nonwoven fabric layer) and a porous film (porous film layer) bonded with each other through a porous adhesive layer (an adhesive layer formed from a fiberized adhesive). The porous adhesive layer is an adhesive layer formed from an adhesive through fiberization of the adhesive. The porous adhesive layer is preferably an adhesive layer formed from a hot-melt adhesive fiberized by spraying (spray coating) and is more preferably an adhesive layer formed from a hot-melt adhesive fiberized by heating and melting the adhesive and spraying the molten adhesive with hot air through curtain spraying to give fibers of adhesive. The adhesive layer, when being a porous adhesive layer formed by fiberization of adhesive, has an advantage of not adversely affecting the air permeability of the air-permeable members. This advantage is more remarkable when the adhesive layer is a porous adhesive layer formed by fiberization of adhesive through spraying.

The average fiber diameter of the porous adhesive layer is preferably from 15 to 500 μm, and more preferably from 20 to 50 μm. If the average fiber diameter is less than 15 μm, the adhesive layer may not give a sufficient bond strength between the nonwoven fabric and the porous film; and if it exceeds 500 μm, heat of the adhesive may propagate to the porous film so as to damage the porous film upon application of the adhesive. The average fiber diameter can be controlled typically by modifying the air flow rate or the distance between the portion to be coated and the curtain spray die during spray coating.

The mass of coating of adhesive to form the porous adhesive layer is preferably from 2 to 10 g/m², and more preferably from 3 to 5 g/m², from the viewpoints typically of adhesiveness and workability in curtain spray. The adhesive, if coated in a mass of less than 2 g/m², may not be coated uniformly and may show insufficient adhesiveness; in contrast, the adhesive, if coated in a mass of more than 10 g/m², may adversely affect the workability.

The porous adhesive layer is formed through fiberization of an adhesive. Though not limited, the adhesive layer is preferably formed (coated) through spraying (spray coating) of an adhesive; is more preferably formed by coating through spraying (spray coating) of a hot-melt adhesive; and is furthermore preferably formed by heating and melting the hot-melt adhesive, and applying the molten adhesive through curtain spray coating with hot air.

Though not critical, the heating temperature (heating-melting temperature) in the curtain spray coating is preferably 180° C. or higher, more preferably from 190° C. to 220° C., and furthermore preferably from 195° C. to 210° C. If the heating temperature is lower than 180° C., the adhesive may show an excessively high viscosity and may not be coated satisfactorily. In contrast, if the heating temperature is excessively high of exceeding 220° C., the curtain spray die may deform due to heat, and this may cause a trouble. In addition, such an excessively heated adhesive may deteriorate to show an insufficient bond strength. Though not critical, the air flow rate is preferably from 200 to 700 liters per minute, and more preferably from 300 to 600 liters per minute. Also though not critical, the air temperature is preferably from 180° C. to 280° C., and more preferably from 200° C. to 260° C.

Properties of Air-Permeable Members for Portable Body Warmers

The air-permeable members for portable body warmers each have a layered structure at least with a nonwoven fabric (a) and a porous film and preferably have a layered structure with the nonwoven fabric (a) and the porous film bonded through an adhesive layer, as described above. The adhesive layer is preferably a porous adhesive layer. The air-permeable members may further include any other nonwoven fabric than the nonwoven fabric (a). Specifically, they may include a composite nonwoven fabric of the nonwoven fabric (a) with another nonwoven fabric. Independently, they may further include any other layer or layers in addition to the nonwoven fabric, adhesive layer, and porous film. Though not limited, preferred examples of the layered structure of the air-permeable members include a layered structure of (nonwoven fabric (a))/(adhesive layer)/(porous film); and a layered structure of (spunlace nonwoven fabric)/(nonwoven fabric (a))/(adhesive layer)/(porous film) [i.e., (composite nonwoven fabric between nonwoven fabric (a) and spunlace nonwoven fabric)/(adhesive layer)/(porous film)].

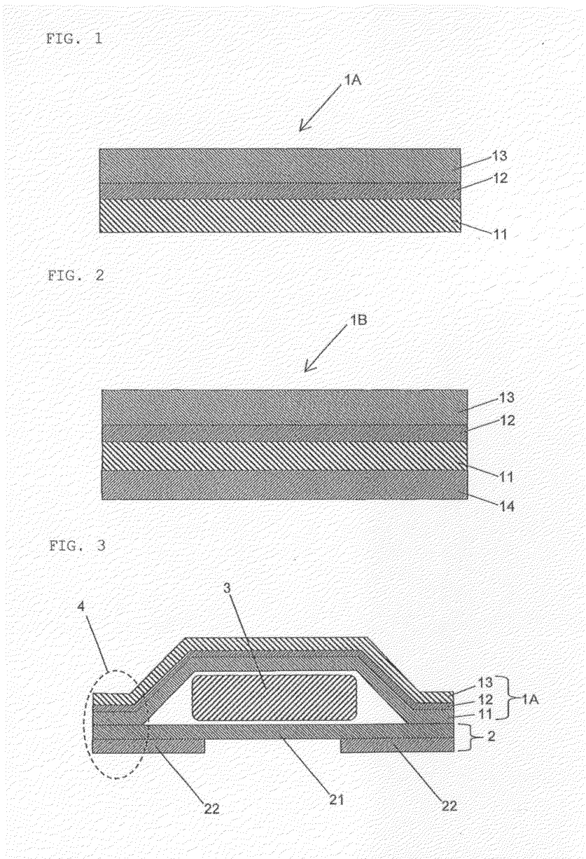

FIG. 1 is a schematic diagram (cross-sectional view) illustrating an air-permeable member for portable body warmers according to an embodiment of the present invention. The air-permeable member 1A includes a nonwoven fabric (a) 11 and a porous film 13 bonded with each other through an adhesive layer 12. FIG. 2 is a schematic diagram (cross-sectional view) illustrating an air-permeable member for portable body warmers according to another embodiment of the present invention. The air-permeable member 1B includes a porous film 13 and a composite nonwoven fabric bonded with each other through an adhesive layer 12, in which the composite nonwoven fabric includes a nonwoven fabric (a) 11 and a spunlace nonwoven fabric (other nonwoven fabric) 14, and the adhesive layer 12 is present on the surface of the nonwoven fabric (a) 11 of the composite nonwoven fabric.

The compressive strengths in a machine direction as measured according to the ring crush method [compressive strength (ring crush method, MD)] of the air-permeable members are from 2 to 5.5 N, preferably from 2 to 5 N, and more preferably from 2.5 to 5 N. The control of the compressive strengths (ring crush method, MD) to 2 N or more helps a portable body warmer using any of the air-permeable members to be resistant to deformation in a machine direction to thereby be produced with higher productivity. In addition, the control of the compressive strengths (ring crush method, MD) to 5.5 N or less helps the portable body warmer to be relatively flexible, and the portable body warmer can be used comfortably without unpleasantness when applied to the clothing or skin. The compressive strengths (ring crush method, MD) can be controlled typically by modifying the production process of the nonwoven fabric, the type of fibers, the mass per unit area, and embossment area ratio, of the nonwoven fabric; and the thickness of the porous film.

Though not critical, the compressive strengths in a transverse direction of the air-permeable members as measured according to the ring crush method is preferably from 1 to 8 N, and more preferably from 2 to 6 N, from the viewpoint of balance in strength as a portable body warmer.

As used herein the term “machine direction” (MD; also referred to as “longitudinal direction” or “lengthwise direction”) refers to the direction of production line of the air-permeable members for portable body warmers. Also as used herein the term “transverse direction” (TD; also referred to as “cross-machine direction” or “crosswise direction”) is a direction orthogonal to the machine direction and a thickness direction.

The “compressive strength in a machine direction as measured according to the ring crush method [compressive strength (ring crush method, MD)]” can be measured using a tensile tester. Specifically, the compressive strength can be measured using the following measurement sample according to the following measurement method. The measurement method will be described in further detail in “evaluation method” later.

Measurement Sample

A cylindrical measurement sample is prepared by sampling a test piece 60 mm long (machine direction) and 60 mm wide (transverse direction) from a sample air-permeable member for portable body warmers, and rounding the test piece with a circumference agreeing the transverse direction. The test piece is a square test piece having two sides agreeing the machine direction and other two sides agreeing the transverse direction of the air-permeable member, respectively. The measurement sample is a cylindrical sample with the machine direction as a height (length) direction and the transverse direction as a circumferential direction and has a height of 60 mm and a perimeter of about 60 mm. In the cylindrical measurement sample, the nonwoven fabric of the air-permeable member faces outward, and the porous film faces inward.

Measurement Method

The measurement is performed while compressing the measurement sample having an initial length of 60 mm in the machine direction at a compressing speed of 300 mm per minute at an ambient temperature of 23° C. and relative humidity of 50% using a tensile tester set in a compression mode. A maximum compressive load at distortions (compression distances) of from 0 to 10 mm is determined, and this is defined as the “compressive strength (ring crush method, MD)”.

The “compressive strength in a transverse direction as measured according to the ring crush method” can be measured by the procedure in the “compressive strength (ring crush method, MD)”, except for interchanging the machine direction and the transverse direction, namely, except for reading “machine direction” for “transverse direction” and reading “transverse direction” for “machine direction”). The “compressive strengths (ring crush method, MD)” of a nonwoven fabric and a porous film can be measured by the procedure in the “compressive strengths (ring crush method, MD)” of the air-permeable members for portable body warmers, except for sampling a test piece from the nonwoven fabric or porous film, instead of from the air-permeable member for portable body warmers.

The surface abrasion resistance of the nonwoven fabric of the air-permeable members for portable body warmers is preferably from Grade 2 to Grade 5, and more preferably from Grade 3 to Grade 5. The abrasion resistance herein is measured in accordance with the Taber abrasion test method specified in JIS L 1906. The abrasion resistance is typically preferably in a range from Grade 3 to Grade 5 when the nonwoven fabric of the air-permeable members is a nonwoven fabric including a polyester nonwoven fabric (a) alone. The control of the abrasion resistance to Grade 2 or higher helps the portable body warmer to be more resistant to nap on its surface (nonwoven fabric surface) upon use, to have good appearance, and to be used comfortably. The abrasion resistance of the air-permeable members can be controlled within the above range by controlling the embossment area ratio within a range of from 5% to 20%

The thicknesses of the air-permeable members are preferably from 100 to 1000 μm, and more preferably from 150 to 700 μm, from the viewpoint of comfort upon use of the resulting portable body warmers.

The air-permeable members for portable body warmers are used as constituent members of portable body warmers. More specifically, they are mainly used as members (hereinafter also referred to as “bag-constituting members”) for constituting a bag housing a heater (heating component or exothermic component). Preferred examples of the portable body warmers include, but are not limited to, adhesive-patch portable body warmers to be applied to clothing, and adhesive-patch portable body warmers to be applied to the skin. Such adhesive-patch portable body warmers (attachable portable body warmers) each have a pressure-sensitive adhesive layer. If the adhesive-patch portable body warmers deform typically during production processes, a release liner for protecting the surface (adhesive face) of the pressure-sensitive adhesive layer deviates from the proper position as a result of deformation to expose the adhesive face, and the exposed adhesive face often causes problems such as attachment to improper portions, thus impairing productivity. Accordingly, the air-permeable members according to the present invention exhibit remarkable advantageous effects to prevent the deformation of portable body warmers of this type. Though not critical, the sizes of the portable body warmers are preferably such that a length in the machine direction is 100 mm or more (for example, 100 to 300 mm) and a length in the transverse direction is 80 mm or more (for example, from 80 to 300 mm). In other words, the portable body warmers are preferably of so-called regular size or larger. More specific examples of the size thereof include a size of 100 mm long (machine direction) and 80 mm wide (transverse direction); a size of 130 mm long (machine direction) and 95 mm wide (transverse direction); and a size of 130 mm long (machine direction) and 100 mm wide (transverse direction). The air-permeable members can exhibit their advantageous effects more remarkably when they are adopted to portable body warmers of regular size or larger size, because such portable body warmers of regular size or larger size are more liable to deform typically during production processes than portable body warmers of so-called mini size. Though not limited, the air-permeable members are more preferably used as a member (so-called “front member”) opposite to a side to be applied to the clothing or skin, so as to ensure satisfactory air permeability.

The air-permeable members for portable body warmers are members each including a nonwoven fabric and a porous film and thereby have air permeability. Though not critical, the air permeability of the air-permeable members is, for example, such that the air resistances of the air-permeable members are preferably 10×10⁴ seconds per 100 cc or less (for example, from 1000 to 10×10⁴ seconds per 100 cc), and more preferably 5×10⁴ seconds per 100 cc or less (for example, from 5000 to 5×10⁴ seconds per 100 cc). The air resistance can be determined according to the Oken tester method specified in JIS P 8117.

There are many situations where compressive force in the machine direction is applied to portable body warmers such as disposable portable body warmers during their production processes and working processes. Examples of such situations include those in the step of transferring the portable body warmer by a push of a jaw; the step of placing the portable body warmers in outer pouches to form individual packages; and the step of bagging the individual packages of portable body warmers typically by ten individual packages. In these situations, a portable body warmer, if having a tendency to deform especially in the machine direction, may suffer from various problems and be produced with insufficient productivity. For example, the deformation of the portable body warmer impedes smooth transfer of the portable body warmer; this also causes the deviation of the release liner for protecting the adhesive face to expose the adhesive face, and the exposed adhesive face of the portable body warmer adheres typically to a part or system in the production line to thereby cause line stop. In addition, the deformation also impedes smooth working in bagging.

In contrast, the productivity of such portable body warmers is successfully improved according to the present invention. Specifically, of constituent members of the portable body warmers, air-permeable members are more liable to deform due to compression in the machine direction than other members such as non-air-permeable pressure-sensitive adhesive sheets for portable body warmers. However, the air-permeable members according to the present invention have improved strengths against compression in the machine direction, and the resulting portable body warmers including the air-permeable members become more resistant to deformation and, especially, become more resistant to deformation caused by compression in the machine direction. In addition, the air-permeable members are controlled to be not excessively rigid, and this helps the portable body warmers to be used comfortably without unpleasantness. Thus, the air-permeable members according to the present invention can give portable body warmers that are usable comfortably and are produced with good productivity.

Portable Body Warmers

Use of the air-permeable members according to the present invention gives portable body warmers such as disposable portable body warmers. The portable body warmers are not especially limited in their structure, as long as including any of the air-permeable members for portable body warmers according to the present invention as a constituent member, but they are preferably, for example, portable body warmers having structures in which the air-permeable member is used as a member (bag-constituting member) constituting a bag for housing or sealing a heater (heating component) therein. The portable body warmers are more preferably portable body warmers (disposable portable body warmers) having structures in which any of the air-permeable members is heat-sealed with another bag-constituting member than the air-permeable member (hereinafter also referred to as “other bag-constituting member”) to form a bag, and a heater is sealed in the bag. Heat sealing to form the bag is preferably performed so that the surface of the porous film of the air-permeable member is brought into contact with the surface of the other bag-constituting member, such as the surface of a base material of a pressure-sensitive adhesive sheet for portable body warmers.

FIG. 3 is a schematic diagram (cross-sectional view) illustrating an exemplary portable body warmer (disposable portable body warmer) using any of the air-permeable members according to the present invention and the other bag-constituting member. The portable body warmer illustrated in FIG. 3 includes a bag, and a heater 3 sealed in the bag. The bag is formed from the air-permeable member 1A and the other bag-constituting member (rear member) 2 by heat-sealing these members in end portions (heat-seal portion 4). The other bag-constituting member 2 is a laminate including a base material 21 and a pressure-sensitive adhesive layer 22. In such a portable body warmer (disposable portable body warmer) including a pressure-sensitive adhesive layer on one side thereof and intended to be applied to an adherend such as a body or clothing as with one illustrated in FIG. 3, the air-permeable member according to the present invention is preferably used at least as a member (so-called front member) opposite to the side to face the adherend, for supplying oxygen to the heater satisfactorily.

The other bag-constituting member can be chosen from among known or common air-permeable or non-air-permeable bag-constituting members without limitation, and examples thereof include bag-constituting members each including a base material and a pressure-sensitive adhesive layer. The other bag-constituting member can also be preferably any of commercially available products such as “Nitotac” supplied by Nitto Lifetec Corporation. The “Nitotac” is a pressure-sensitive adhesive sheet for portable body warmers and is a laminate of a heat-sealable polyolefin base material and a styrene-isoprene-styrene block copolymer (SIS) pressure-sensitive adhesive layer.

The base material in the other bag-constituting member preferably includes at least one of a heat-sealable layer, a fibrous layer (for example, nonwoven fabric layer), and a film layer. More specific examples of the base material include a laminate of a heat-sealable layer (inclusive of heat-sealable film layer) and a fibrous layer; and a laminate of a heat-sealable layer and a non-heat-sealable film layer.

Examples of a nonwoven fabric usable in the nonwoven fabric layer include known or common nonwoven fabrics including nonwoven fabrics made from natural fibers and nonwoven fabrics made from synthetic fibers, such as nylon nonwoven fabrics, polyester nonwoven fabrics, polyolefin nonwoven fabrics, and rayon nonwoven fabrics. The nonwoven fabric is not limited in production process and can be any of nonwoven fabrics produced by spunbonding (spunbond nonwoven fabrics) and nonwoven fabrics produced by spunlacing (spunlace nonwoven fabrics). The nonwoven fabric may have a single-layer structure or multilayer structure. The fiber diameter, fiber length, mass per unit area, and other conditions of the nonwoven fabric are not especially limited, but the nonwoven fabric is preferably one having a mass per unit area of approximately from 20 to 150 g/m², from the viewpoints of workability and cost. The nonwoven fabric may contain fibers of one type alone or contain fibers of two or more types in combination.

The heat-sealable layer is a layer having heat sealability and preferably contains any of the resins listed in the porous film. The heat-sealable layer may have a single-layer structure or multilayer structure.

The film layer can be any of film layers commonly used. Exemplary resins usable to constitute the film layer include polyester resins and olefinic resins. Among them, olefinic resins are preferably used from the viewpoints of price and flexibility. The olefinic resins can for example be any of the resins listed in the porous film. The film layer may have a single-layer structure or multilayer structure including two or more layers. Independently, the film layer may be a non-oriented film or a monoaxially or biaxially oriented film, but is preferably a non-oriented film.

Though not critical, the thickness of the base material is, for example, about from 10 to 500 μm, preferably about from 12 to 200 μm, and furthermore preferably about from 15 to 100 μm. The base material may have been subjected to a treatment such as backing and antistatic treatment according to necessity.

The pressure-sensitive adhesive layer in the other bag-constituting member has the function of affixing the bag to the adherend upon use. Though not limited, exemplary pressure-sensitive adhesives for constituting the pressure-sensitive adhesive layer include rubber pressure-sensitive adhesives, urethane pressure-sensitive adhesives (acrylic urethane pressure-sensitive adhesives), acrylic pressure-sensitive adhesives, silicone pressure-sensitive adhesives, polyester pressure-sensitive adhesives, polyamide pressure-sensitive adhesives, epoxy pressure-sensitive adhesives, vinyl alkyl ether pressure-sensitive adhesives, fluorine-containing pressure-sensitive adhesives, and other known pressure-sensitive adhesives. Each of different pressure-sensitive adhesives can be used alone or in combination. Among them, rubber and urethane (acrylic urethane) pressure-sensitive adhesives are especially preferred.

Examples of the rubber pressure-sensitive adhesives include rubber pressure-sensitive adhesives containing any of natural rubbers and synthetic rubbers as a base polymer. Exemplary rubber pressure-sensitive adhesives containing a synthetic rubber as a base polymer include styrenic rubbers (also called styrenic elastomers) such as styrene-butadiene (SB) rubbers, styrene-isoprene (SI) rubbers, styrene-isoprene-styrene block copolymer (SIS) rubbers, styrene-butadiene-styrene block copolymer (SBS) rubbers, styrene-ethylene-butylene-styrene block copolymer (SEBS) rubbers, styrene-ethylene-propylene-styrene block copolymer (SEPS) rubbers, styrene-ethylene-isoprene-styrene block copolymer (SIPS) rubbers, and styrene-ethylene-propylene block copolymer (SEP) rubbers; polyisoprene rubbers; reclaimed rubbers; butyl rubbers (isobutylene-isoprene rubbers); polyisobutylenes; and modified substances derived from these rubbers. Among them, styrenic elastomer pressure-sensitive adhesives are preferred, of which SIS and SBS are more preferred. Each of these can be used alone or in combination as a mixture.

The urethane pressure-sensitive adhesives can be any known or common urethane pressure-sensitive adhesives without limitation, but preferred examples thereof include the urethane pressure-sensitive adhesives exemplified in Japanese Patent No. 3860880 and Japanese Unexamined Patent Application Publication (JP-A) No. 2006-288690. Among them, acrylic urethane pressure-sensitive adhesives composed of isocyanate/polyester polyols are more preferred. Of the acrylic urethane pressure-sensitive adhesives, preferred are expandable (foamable) pressure-sensitive adhesives containing bubbles, from the viewpoint of reducing skin irritation when the portable body warmer is applied directly to the skin. Such expandable pressure-sensitive adhesives can be prepared, for example, by a process of compounding a known or common blowing agent into a pressure-sensitive adhesive.

The pressure-sensitive adhesive for use herein can be any of pressure-sensitive adhesives of different types (properties), such as pressure-sensitive adhesives having heat curability (heat-curable pressure-sensitive adhesives), in which crosslinks or other structures are formed upon the application of heat, whereby the adhesives are cured; and pressure-sensitive adhesives having curability by the action of active energy rays (active-energy-ray-curable pressure-sensitive adhesives), in which crosslinks or other structures are formed upon the application of active energy rays, whereby the adhesives are cured. Among them, active-energy-ray-curable pressure-sensitive adhesives are preferred, because they can be free from solvents and are thereby not excessively impregnated into a nonwoven fabric or porous base material. The heat-curable pressure-sensitive adhesives may further contain one or more of crosslinking agents and polymerization initiators for exhibiting heat curability as appropriate. The active-energy-ray-curable pressure-sensitive adhesives may further contain one or more of crosslinking agents and photoinitiators for exhibiting curability by the action of active energy rays as appropriate.

The pressure-sensitive adhesive layer may be protected by a known or common release liner (also called release film or separator) before use.

The portable body warmers are housed in outer pouches, respectively, and are marketed as portable body warmer products. A base material (outer pouch base material) for use to constitute the outer pouches is not especially limited, and examples thereof include plastic base materials; fibrous base materials, such as nonwoven fabric base materials and woven fabric base materials each made from a fiber of every kind; and metallic base materials, such as metal foil base materials each made from a metallic component of every kind. Among them, plastic base materials are preferably used as the base materials. Examples of the plastic base materials include polyolefin base materials such as polypropylene base materials and polyethylene base materials; polyester base materials such as poly(ethylene terephthalate) base materials; styrenic base materials including polystyrene base materials, and styrenic copolymer base materials such as acrylonitrile-butadiene-styrene copolymer base materials; amide resin base materials; and acrylic resin base materials. The outer pouch base material may have a single layer structure or a multilayer structure. Though not critical, the thickness of the outer pouch is preferably from 30 to 300 μm.

In a preferred embodiment, the outer pouch has a layer (gas barrier layer) having gas barrier properties for inhibiting the permeation of gaseous components such as oxygen gas and water vapor. The gas barrier layer is not particularly limited, and examples thereof include oxygen-barrier resin layers such as those made from poly(vinylidene chloride) resins, ethylene-vinyl alcohol copolymers, poly(vinyl alcohol)s, and polyamide resins; water-vapor-barrier resin layers such as those made from polyolefins and poly(vinylidene chloride)s; and oxygen-barrier and/or water-vapor-barrier inorganic compound layers such as those made from elementary metals such as aluminum, and metallic compounds including metal oxides such as silicon oxide and aluminum oxide. The gas barrier layer may be a single layer (e.g., it may be the outer pouch base material itself) or a multilayer laminate.

The outer pouch may be a pouch of any form and structure, such as so-called “four-sided sealed pouch (four side seal pouch),” “three-sided sealed pouch (three side seal pouch),” “pillow style pouch,” “stand-up pouch” (“standing pouch”), or “gusseted pouch.” In a preferred embodiment, the outer pouch is a four-sided sealed pouch. The outer pouch may be prepared using an adhesive, but it is preferably prepared by heat sealing (thermofusing) as typically in a four-sided heat-sealed pouch.

The present invention will be illustrated in further detail with reference to several examples below. It should be noted, however, these examples are never construed to limit the scope of the present invention.

### Example 1

### Nonwoven Fabric

A nonwoven fabric used herein was a spunbond nonwoven fabric prepared from PET fibers through spunbonding and having an embossment area ratio of 11% and a mass per unit area of 30 g/m².

The nonwoven fabric had an area per single embossment pattern of 1.2 mm².

Porous Film

A material mixture was prepared by melting and kneading, at 180° C., 100 parts by weight of a linear low-density polyethylene (having a weight-average molecular weight Mw of 6×10⁴ and a MFR (190° C.) of 2.3 g/10 min.), 40 parts by weight of an ethylene-α-olefin copolymer (having a weight-average molecular weight Mw of 11.2×10⁴ and a MFR (190° C.) of 3.6 g/10 min.), 140 parts by weight of calcium carbonate (having an average particle diameter of 1.1 μm), 1 part by weight of stearic acid, and 1 part by weight of an antioxidant.

The material mixture was subjected to melting/extrusion according to the T-die process, the extruded article was monoaxially drawn in a longitudinal direction (machine direction) at a drawing temperature of 100° C. and a draw ratio of 4 times, and thereby yielded a polyethylene porous film 70 μm thick.

Adhesive

An adhesive used herein was a hot-melt polyamide adhesive.

### Air-Permeable Member

Pellets of the polyamide adhesive were charged into an applicator tank, melted at 190° C., and applied to one side of the nonwoven fabric through curtain spray coating to form a fibrous porous adhesive layer having a basis weight of 5 g/m². The curtain spray coating was performed at an air temperature of 230° C. and an air flow rate of 550 liters per minute.

Next, the polyethylene porous film was affixed onto the adhesive layer and thereby yielded an air-permeable member. The air-permeable member had a compressive strength (ring crush method, MD) of 4.1 N and an abrasion resistance of Grade 3.5.

The preparation of the polyethylene porous film and the preparation of the porous adhesive layer and the affixation were performed in-line in the same production line.

### Example 2

An air-permeable member was prepared by the procedure of Example 1, except for switching from the nonwoven fabric to a spunbond nonwoven fabric prepared from PBT fibers through spunbonding and having an embossment area ratio of 13%, a mass per unit area of 30 g/m², and an area per single embossment pattern of 0.6 mm². The air-permeable member had a compressive strength (ring crush method, MD) of 4.3 N and an abrasion resistance of Grade 4.

### Example 3

A nonwoven fabric used herein was a composite nonwoven fabric of a spunbond nonwoven fabric and a spunlace nonwoven fabric, in which the spunbond nonwoven fabric had been prepared from PET fibers through spunbonding and had an embossment area ratio of 11%, a mass per unit area of 20 g/m², and an area per single embossment pattern of 1.2 mm²; and the spunlace nonwoven fabric had been prepared from PET fibers through spunlacing and had a mass per unit area of 20 g/m².

An air-permeable member was prepared by the procedure of Example 1, except for switching from the nonwoven fabric to the composite nonwoven fabric. In the preparation, an adhesive layer was formed on the surface of the spunbond nonwoven fabric of the composite nonwoven fabric, and the porous film was affixed to the adhesive layer.

The air-permeable member had a compressive strength (ring crush method, MD) of 2.9 N and an abrasion resistance of Grade 5.

### Comparative Example 1

An air-permeable member was prepared by the procedure of Example 1, except for switching from the nonwoven fabric to a spunbond nonwoven fabric prepared from PET fibers through spunbonding and having an embossment area ratio of 11%, a mass per unit area of 100 g/m², and an area per single embossment pattern of 1.2 mm². The air-permeable member had a compressive strength (ring crush method, MD) of 9.3 N and an abrasion resistance of Grade 3.5.

### Comparative Example 2

An air-permeable member was prepared by the procedure of Example 1, except for switching from the nonwoven fabric to a spunbond nonwoven fabric prepared from PET fibers through spunbonding and having an embossment area ratio of 28%, a mass per unit area of 30 g/m², and an area per single embossment pattern of 0.3 mm². The air-permeable member had a compressive strength (ring crush method, MD) of 5.7 N and an abrasion resistance of Grade 5.

### Comparative Example 3

An air-permeable member was prepared by the procedure of Example 1, except for switching from the nonwoven fabric to a spunbond nonwoven fabric prepared from nylon fibers through spunbonding and having an embossment area ratio of 14%, a mass per unit area of 35 g/m², and an area per single embossment pattern of 0.2 mm². The air-permeable member had a compressive strength (ring crush method, MD) of 1.0 N and an abrasion resistance of Grade 5.

### Comparative Example 4

An air-permeable member was prepared by the procedure of Example 1, except for switching from the nonwoven fabric to a spunlace nonwoven fabric prepared from PET fibers through spunlacing and having a mass per unit area of 30 g/m². The air-permeable member had a compressive strength (ring crush method, MD) of 0.6 N and an abrasion resistance of Grade 5.

Evaluations

The following evaluations were performed on the air-permeable members prepared in the examples and comparative examples. The embossment area ratios of the nonwoven fabrics were measured in the following manner.

The evaluation results are shown in Table 1.

(1) Embossment Area Ratio

The surface of a sample nonwoven fabric was observed with a microscope (trade name “VHX-200” supplied by Keyence Corporation, of a magnification of 25 times), the percentage by area (area percent) of embossed portion in a measurement region (10 mm long (machine direction) and 10 mm wide (transverse direction)) was determined as the embossment area ratio according to the following equation:

embossment area ratio (%)=(area of embossed portion; in mm²)/100×100

The measurements were performed a total of five times (n=5) on one side of the nonwoven fabric, and the average of five measurements was defined as the embossment area ratio of the nonwoven fabric.

(2) Compressive Strength (Ring Crush Method, MD) Measurement Sample

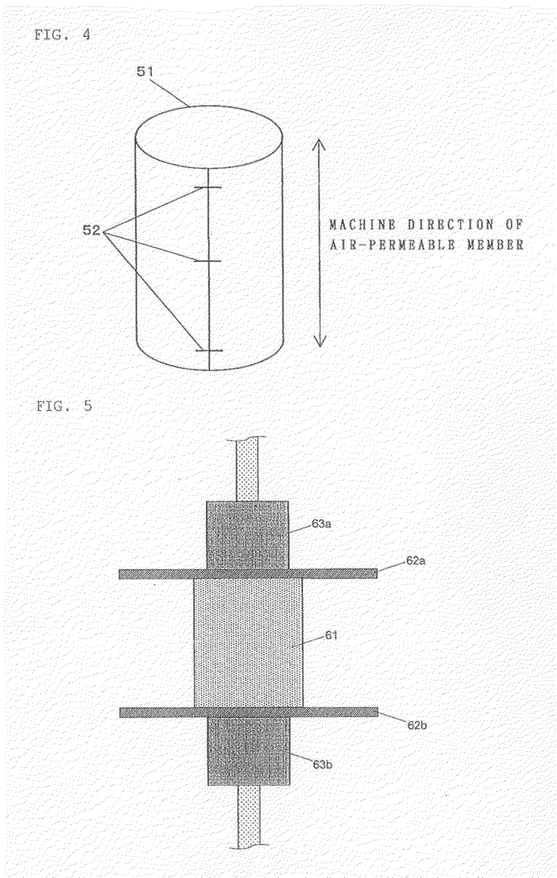

Test pieces 60 mm long (machine direction) and 60 mm wide (transverse direction) were sampled from the air-permeable members prepared in the examples and comparative examples. The test pieces were each a square test piece having two sides agreeing the machine direction and other two sides agreeing the transverse direction. The test pieces were rounded cylindrically (with a circumferential direction agreeing the transverse direction) so that the nonwoven fabric of the air-permeable member faced outward and the porous film faced inward; and both end portions were stapled at three points using a stapler to give cylindrical measurement samples (FIG. 4). The measurement samples were each a cylindrical sample with a height (length) direction agreeing the machine direction and with a circumferential direction agreeing the transverse direction and had a height of 60 mm and a perimeter of about 60 mm.

FIG. 4 is a schematic diagram (perspective view) illustrating the cylindrical measurement sample. In FIG. 4, the reference numerals “51” stands for an air-permeable member for portable body warmers (test piece), and “52” stands for a staple. The staples 52 are located in the height direction at heights of about 5 mm, about 30 mm, and about 55 mm, from the bottom.

Measurement Method

The measurement as a compression test was performed at an ambient temperature of 23° C. and relative humidity of 50% using a tensile tester (trade name “RTC-1210A” supplied by A&D Company Ltd.). Specifically, the measurement was performed while compressing the measurement sample having an initial length of 60 mm in the machine direction according to the compression mode of the tester at a compressing speed of 300 mm per minute. A maximum compressive load at distortions (compression distances) of from 0 to 10 mm was determined, and this was defined as the “compressive strength (ring crush method, MD)”.

FIG. 5 is an explanatory drawing schematically illustrating how to set the measurement sample in the tensile tester in the measurement of the compressive strength (ring crush method, MD) and is a schematic front view showing a region around grips (chucks) of the tensile tester. With reference to FIG. 5, the measurement was performed such that two stainless steel (SUS) sheets 62a and 62b each having a size of 100 mm long and 100 mm wide were arranged horizontally between the tips of upper and lower grips (chucks) 63a and 63b of the tensile tester, respectively; the measurement sample 61 was placed between the two SUS sheets 62a and 62b and was compressed between the two SUS sheets 62a and 62b.

(3) Abrasion Resistance

Round measurement samples 130 mm in diameter were sampled from the air-permeable members prepared in the examples and comparative examples.

The abrasion resistances of the measurement samples were measured using a Taber abrasion tester according to the Taber method specified in JIS L 1906.

(4) Resistance to Deformation of Portable Body Warmer

Preparation of Portable Body Warmer

Measurement samples 130 mm long (machine direction) and 95 mm wide (transverse direction) were cut out from the air-permeable members prepared in the examples and comparative examples. As another bag-constituting member, a pressure-sensitive adhesive sheet for portable body warmers (trade name “Nitotac” supplied by Nitto Lifetec Corporation) 130 mm long and 95 mm wide was used.

Each measurement sample and the pressure-sensitive adhesive sheet were laid on each other so that the surface of the porous film of the measurement sample faced a surface (film side) of the pressure-sensitive adhesive sheet opposite to the pressure-sensitive adhesive layer; the two members were heat-sealed in three sides to form a bag with an opening in the residual one side; a heater of a commercially available portable body warmer was placed into the bag from the opening; and the opening was heat-sealed to give a portable body warmer.

The heat sealing was performed under conditions of a temperature of 120° C. for 0.5 second with a sealing width of 5 mm. The heat sealing was conducted at end portions of the laminate.

Evaluation of Resistance to Deformation

The resistance to deformation of each of the above-prepared portable body warmers was evaluated according to the following criteria, based on the workability when the portable body warmer was packed into an outer pouch by hand. The outer pouch was one made from a polystyrene, having a size of 165 mm in length and 120 mm in width, and having been heat-sealed with a sealing width of 10 mm in three sides, i.e., two sides in the length direction and one side in the width direction.

A sample portable body warmer was evaluated as being good to deformation (“Good”) when the portable body warmer could be smoothly packed into the outer pouch without deformation and curling of the portable body warmer at the four corners and without curling up and pop-off of the release liner (release paper) of the pressure-sensitive adhesive sheet. In contrast, a sample portable body warmer was evaluated as being apt to deform (“Poor”) when the portable body warmer could not be smoothly packed into the outer pouch while suffering from deformation and/or curling of the portable body warmer at the four corners and/or suffering from curling up and/or pop-off of the release liner of the pressure-sensitive adhesive sheet.

(5) Feel (Feeling) Upon Use of Portable Body Warmer

(Presence or Absence of Unpleasantness upon Use of Portable Body Warmer)

A series of portable body warmers was prepared by the procedure of “(4) resistance to deformation of portable body warmer” using the air-permeable members prepared in the examples and comparative examples.

Feel upon use was evaluated on each of the above-prepared portable body warmers. Specifically, each portable body warmer was affixed to a tee shirt on the back so that the machine direction of the air-permeable member was in parallel with the backbone, and a feel (impression) during use was evaluated.

A sample portable body warmer giving no unpleasantness was evaluated as giving comfortable feel upon use (Good); and in contrast, a sample portable body warmer giving stiff feel and unpleasantness typically when the back was bent was evaluated as giving uncomfortable feel upon use (Poor).

TABLE 1

| | | | | Air-permeable | | | |
|---|---|---|---|---|---|---|---|
| | | | | member | | Portable body warmer | |
| | | | | | Compressive | | Feel upon use |
| | Nonwoven fabric | | | | strength | | (presence or |
| | | Mass per | Embossment | Abrasion | (ring crush | | absence of |
| | | unit area | area ratio | resistance | method, | Resistance to | unpleasantness |
| | Material/process | (g/m²) | (%) | (Grade) | MD) (N) | deformation | during use) |
| Example 1 | PET/spunbonding | 30 | 11 | 3.5 | 4.1 | Good | Good |
| Example 2 | PBT/spunbonding | 30 | 13 | 4 | 4.3 | Good | Good |
| Example 3 | front side: | PET/ | PET/ | 5 | 2.9 | Good | Good |
| | PET/spunlacing | spunlacing: 20 | spunlacing: - | | | | |
| | porous film side: | PET/ | PET/ | | | | |
| | PET/spunbonding | spunbonding: 20 | spunbonding: 11 | | | | |
| Com. Ex. 1 | PET/spunbonding | 100 | 11 | 3.5 | 9.3 | Good | Poor |
| Com. Ex. 2 | PET/spunbonding | 30 | 28 | 5 | 5.7 | Good | Poor |
| Com. Ex. 3 | nylon/spunbonding | 35 | 14 | 5 | 1.0 | Poor | Good |
| Com. Ex. 4 | PET/spunlacing | 30 | — | 5 | 0.6 | Poor | Good |

As is demonstrated in Table 1, the air-permeable members according to the present invention (Examples 1 to 3) gave portable body warmers which were resistant to deformation and were comfortably usable without unpleasantness. In contrast, the air-permeable members having compressive strengths (ring crush method, MD) of more than 5.5 N (Comparative Examples 1 and 2) gave portable body warmers which gave unpleasantness upon use and were not usable comfortably. The air-permeable members having compressive strengths (ring crush method, MD) of less than 2 N (Comparative Examples 3 and 4) gave portable body warmers which were apt to deform and showed insufficient productivity.

1. An air-permeable member for portable body warmers, the member comprising a layered structure with at least a nonwoven fabric and a porous film,
wherein the nonwoven fabric is a spunbonded nonwoven fabric having an embossment area ratio of from 5% to 20% and a mass per unit area of from 10 to 80 g/m²,
wherein the porous film has a thickness of from 30 to 200 μm,
wherein the air-permeable member has a compressive strength in a machine direction of from 2 to 5.5 newtons (N) as measured according to a ring crush method, and
wherein the compressive strength in a machine direction as measured according to the ring crush method is a maximum compressive load determined by sampling a 60-mm square test piece from the air-permeable member for portable body warmers, the test piece having sides corresponding to the machine direction and transverse direction of the air-permeable member, respectively; rounding the test piece with a circumference agreeing the transverse direction to give a cylindrical measurement sample; compressing the cylindrical measurement sample in the machine direction at an ambient temperature of 23° C. and relative humidity of 50% at a compressing speed of 300 mm per minute using a tensile tester according to a compression mode; and measuring compressive loads at distortions (compression distances) of from 0 to 10 mm to determine the maximum compressive strength.

2. An air-permeable member for portable body warmers, the member comprising a layered structure with at least a nonwoven fabric and a porous film,
wherein the nonwoven fabric is a composite nonwoven fabric including a spunbonded nonwoven fabric and a spunlace nonwoven fabric, the spunbonded nonwoven fabric having an embossment area ratio of from 5% to 20% and a mass per unit area of from 10 to 80 g/m²,
wherein the porous film has a thickness of from 30 to 200 μm,
wherein the air-permeable member has a compressive strength in a machine direction of from 2 to 5.5 N as measured according to a ring crush method, and
wherein the compressive strength in a machine direction as measured according to the ring crush method is a maximum compressive load determined by sampling a 60-mm square test piece from the air-permeable member for portable body warmers, the test piece having sides corresponding to the machine direction and transverse direction of the air-permeable member, respectively; rounding the test piece with a circumference agreeing the transverse direction to give a cylindrical measurement sample; compressing the cylindrical measurement sample in the machine direction at an ambient temperature of 23° C. and relative humidity of 50% at a compressing speed of 300 mm per minute using a tensile tester according to a compression mode; and measuring compressive loads at distortions (compression distances) of from 0 to 10 mm to determine the maximum compressive strength.

3. The air-permeable member for portable body warmers, according to claim 1, wherein the nonwoven fabric comprises a polyester nonwoven fabric.

4. The air-permeable member for portable body warmers, according to claim 2, wherein the nonwoven fabric comprises a polyester nonwoven fabric.
